(19) United States
(12) Patent Application Publication
(10) Pub. No.: US 2025/0385707 A1
(43) Pub. Date: Dec. 18, 2025

(54) FRONT-END COMMUNICATION UNIT FOR AN IMPLANT COMMUNICATION SYSTEM AND METHOD FOR OPERATING THE FRONT-END COMMUNICATION UNIT

(71) Applicant: BIOTRONIK SE & Co. KG, Berlin (DE)

(72) Inventors: Hendrik DEUTSCHMANN, Berlin (DE); Lisa BILLER, Berlin (DE)

(73) Assignee: BIOTRONIK SE & Co. KG, Berlin (DE)

(21) Appl. No.: 18/879,136

(22) PCT Filed: Jun. 14, 2023
(86) PCT No.: PCT/EP2023/065891

§ 371 Date: Dec. 26, 2024

(30) Foreign Application Priority Data
Jul. 11, 2022 (EP) 22184099.4

(51) Int. Cl. H04B 1/40; G10L 15/22; G16H 40/67

(52) U.S. Cl. CPC H04B 1/40; G10L 15/22; G10L 2015/223; G16H 40/67

(57) ABSTRACT

A front-end communication unit for an implant communication system comprising a communication interface configured to read out data from an implantable medical device, and a control unit communicatively connected to the communication interface, the control unit being configured to transfer the data read out by the communication interface to a back-end communication unit, and wherein the front-end communication unit is configured to receive audio input and provide audio output. Further, a computer implemented method for operating a front-end communication unit for an implant communication system, and wherein the communication interface comprises an LTE and/or 5G-interface, configured to receive incoming voice calls and to initiate outgoing voice calls is also disclosed.

### CROSS-REFERENCE TO RELATED APPLICATIONS

This application is the United States National Phase under 35 U.S.C. § 371 of PCT International Patent Application No. PCT/EP2023/065891, filed on Jun. 14, 2023, which claims the benefit of European Patent Application No. 22184099.4, filed on Jul. 11, 2022, the disclosures of which are hereby incorporated by reference herein in their entireties.

### TECHNICAL FIELD

The present invention relates to a front-end communication unit for an implant communication system.

Furthermore, the present invention relates to a computer implemented method for operating a front-end communication unit for an implant communication system.

### BACKGROUND

U.S. Publication No. 2020/0357513 A1 discloses techniques for remote monitoring of a patient and corresponding medical device(s). The remote monitoring comprises providing an interactive session configured to allow a user to navigate a plurality of sub sessions, determining a first set of data items in accordance with a first sub session, the first set of data items including the image data, determining a second set of data items in accordance with a second sub session of the interactive session, determining, based at least in part on the first set of data items and the second set of data items, an abnormality, and outputting a post-implant report of the interactive session.

U.S. Publication No. 2016/0232010 A1 relates to the provisioning and tracking of software installed in mobile devices. More particularly, it relates to the automatic download and installation of software and/or firmware in mobile devices, and for the tracking, ordering, and configuration of electronic devices.

U.S. Publication No. 2014/0018779 A1 is directed to a telemedicine care system. In one possible configuration and by non-limiting example, the telemedicine care system includes a patient care unit that provides audio and/or video conferencing capabilities and diagnostic instruments for remote examination of and interaction with a patient by a healthcare professional.

U.S. Publication No. 2017/0049339 A1 describes the provision of a stethoscope front-end recorder device (also referred to as Sleeve) for the chest piece of a stethoscope, which is easily installed and removed. The Sleeve covers the circumference of the chest piece of a stethoscope. The Sleeve contains sensors for acquiring biosensor parameters such as electrocardiogram (ECG), body temperature, heartbeat, heart rhythm, heart rate variability (HRV), heart rate turbulence (HRT), heart sounds, respiration, heart murmur, heart rhythm, heart failure and blood flow.

International Publication No. WO 2007/070669 A2 discloses a patient management device for portably interfacing with a plurality of implantable medical devices and method thereof is presented. Permission to interrogate one or more implantable medical devices is authenticated. Patient device data is individually exchanged through interrogation of at least one authenticated implantable medical device through short range telemetry. External device data is exchanged via communication with at least one external device through long range telemetry. At least one of the patient device and external device data is maintained contemporaneously to execution of operations to perform one or more of relay, processing, and outputting of the patient device and external device data subsequent to the interrogation of the implantable medical device.

Conventional implant systems hence comprise a programmer capable of reading out data from and writing data to an implantable medical device. Such proprietary programming devices can also be used solely for reading out data from the implantable medical device.

If a patient wearing an implantable medical device is admitted to a hospital and/or medical practice due to an acute medical condition data is read out from the implantable medical device by means of said programmer by a trained expert and/or medical practitioner in order to evaluate medical parameters of the patient recorded by the implantable medical device as well as technical parameters of the implantable medical device in order to evaluate if the implantable medical device is operating normally or whether a technical issue is present.

Programmers however are not universally available at all medical facilities such as hospitals and/or medical practices. Furthermore, such programmers may be difficult to use for untrained medical staff, acceptance of such devices is generally limited. In addition, programming devices are equipped with many functions which, if used incorrectly, can adversely affect the health of the patient/implant carrier.

The present disclosure is directed toward overcoming one or more of the above-mentioned problems, though not necessarily limited to embodiments that do.

### SUMMARY

It is therefore an object of the present invention to provide an improved front-end communication unit for an implant communication system that is usable not only by medical practitioners but also by untrained medical staff and is generally widely accepted and universally available.

At least the object is solved by a front-end communication unit for an implant communication system having the features of claim 1.

Furthermore, at least the object is solved by a method for operating a front-end communication unit for an implant communication system having the features of claim 14.

In addition, at least the object is solved by a computer program with program code to perform the method according to the present invention when the computer program is executed on a computer having the features of claim 15.

Further developments and advantageous embodiments are defined in the dependent claims.

The present invention provides a front-end communication unit for an implant communication system comprising a communication interface configured to (at least) read out data from an implantable medical device, and a control unit communicatively connected to the communication interface, the control unit being configured to transfer the data read out by the communication interface to a back-end communication unit, and wherein the front-end communication unit is configured to receive audio input and provide audio output.

Additionally, the communication device may be configured to write (or program) data into the implantable medical device.

Furthermore, the present invention provides a computer implemented method for operating a front-end communication unit for an implant communication system.

The method comprises reading out data from an implantable medical device by means of a communication interface and providing a control unit communicatively connected to the communication interface.

In addition, the method comprises transferring the data read out by the communication interface to a back-end communication unit by means of the control unit and receiving audio input and providing audio output by means of the front-end communication unit.

The control unit comprises a housing, preferably a screen, data processing and communication means and means to receive audio input and provide audio output. The control unit is not a smartphone, but preferably comprises communication interfaces that enable connecting to a range of communication devices such as Wi-Fi routers and/or smartphones.

The control unit is preferably connected to the communication interface by means of a cable or Bluetooth connection. The communication interface is preferably a device that is placed on the skin of the patient to read-out data from the implantable medical device. Alternatively, both the control unit and the communication interface may be comprised in a single device that may communicate with the implantable medical device by means of a wireless, preferably a Bluetooth, connection.

An idea of the present invention is to provide a front-end communication unit for an implant communication system comprising acoustic/audio feedback. By integrating an acoustic interface for interaction, advantageously the use cases of the device can be extended.

Furthermore, the device is better handleable by a wider range of users, is less error prone and, in general, the customer/user satisfaction and compliance with the device and associated workflows can be increased.

Moreover, the device provides an improved handling for impaired/handicapped persons.

Further, due to steady increasing means to interact with technology and increasing numbers of digital natives becoming medical personal, users can advantageously use the front-end communication unit in the same way they are used to use consumer devices, i.e., with audio support, speech recognition and connected devices.

The implantable medical device may be formed by a purely therapeutic implant (implant with therapeutic functions), a purely diagnostic implant (implant with diagnostic functions) and/or an implant with both therapeutic and diagnostic functions.

An example of a purely therapeutic implant/implantable medical device is, e.g., a stimulator/electrode for deep brain stimulation. The therapy consists of the delivery of pulse trains without collecting diagnostic data from the stimulator.

An example of a purely diagnostic implant is, e.g., a cardiac rhythm monitor. The diagnostic function consists of continuous recording of the patient's ECG and automatic evaluation of abnormalities of the heart rhythm. If such are detected, an ECG recording is stored and typically automatically transmitted to a remote monitoring system.

An example of an implant with both therapeutic and diagnostic functions is, e.g., a cardiac pacemaker. The pacemaker is typically implanted subcutaneously in the upper right thoracic region and the electrode is placed in the patient's heart via a large vein. The therapeutic function consists of delivering stimulation pulses to trigger a cardiac action, provided there is no spontaneous cardiac action in the patient. The diagnostic function consists, for example, in the continuous recording of the patient's ECG and automatic evaluation of abnormalities of the heart rhythm. If such are detected, an ECG recording is stored and typically automatically transmitted to a remote monitoring system.

According to an aspect of the present invention, the front-end communication unit comprises an audio processing unit configured to process the audio input by means of a voice recognition algorithm, wherein the audio input is configured to process user commands and/or user questions directed to the front-end communication unit.

This advantageously enables the user to ask questions such as “what is the next step?”, “has the data been transmitted successfully?” or “what is the battery status?”. Furthermore, it enables the user to instruct the device to carry out actions such as “search for an update”, “install the update” or “contact the service center”.

According to a further aspect of the present invention, the audio processing unit is configured, whenever a user command and/or user question directed to the front-end communication unit is recognized by the voice recognition algorithm, to perform a predefined function associated with the user command and/or user question. This advantageously provides a more efficient user interaction with the front-end communication unit.

According to a further aspect of the present invention, the audio output comprises audible status information of the front-end communication unit and/or the implantable medical device, and wherein the audio output further comprises audible instructions directed to a user of the front-end communication unit. The audio output thus enables the user, i.e., the healthcare practitioner to perform other tasks such as patient care at the same time than interacting with the front-end communication unit.

According to a further aspect of the present invention, the communication interface comprises a mobile interface, in particular an LTE and/or 5G-interface, configured to receive incoming voice calls and to initiate outgoing voice calls.

According to a further aspect of the present invention, the communication interface further comprises a mobile interface different from the LTE and/or 5G-interface (e.g., a 6G-interface), configured to receive incoming voice calls and to initiate outgoing voice calls.

The possibility of receiving incoming calls advantageously enables a field representative to directly contact the operator of the device without the need to know who it is. The possibility of initiating outgoing calls enables to contact the service center of the healthcare provider in order to get assistance on specific issues.

According to a further aspect of the present invention, the communication interface comprises a Bluetooth-interface configured to connect to the implantable medical device and/or to connect to a mobile communication device, in particular a smartphone.

The operator of the device can thus also make outgoing calls and or receive incoming calls by connecting to the mobile communication device. In addition, patient medical data may thus in this way be sent to a back-end communication unit of the implant communication system.

According to a further aspect of the present invention, the communication interface comprises a WiFi-interface configured to transmit the data read out from the implantable medical device to a back-end communication unit of the implant communication system.

Said WiFi-interface thus provides an additional data communication interface which advantageously provides another communication pathway in addition to Bluetooth and mobile communication.

According to a further aspect of the present invention, the front-end communication unit is configured, upon a change of information displayed on a screen of the front-end communication unit, to generate an audio output corresponding to the information displayed on the screen of the front-end communication unit. The user is thus advantageously always notified when there is a change of information displayed on the screen of the front-end communication unit.

According to a further aspect of the present invention, the front-end communication unit is configured, upon a status change of the front-end communication unit, to generate an audio output corresponding to the status change of the front-end communication unit. For example, “device switched on—welcome” is an example of a status change of the front-end communication unit. Another example of a status change could be “battery charging”.

According to a further aspect of the present invention, the front-end communication unit is configured, when a user interaction with the front-end communication unit is required, to generate an audio output corresponding to the required user interaction with the front-end communication unit. An example of a user interaction requirement is the notification “battery low—please charge.” and/or “device goes in power save mode—please press button xyz to return to normal operation”.

According to a further aspect of the present invention, the front-end communication unit is configured to receive and process audio input whenever there is no audio output. This enables an easy and efficient hands-free interaction of the user with the front-end communication unit.

According to a further aspect of the present invention, in case of an operating error and/or technical malfunction of the front-end communication unit, the front-end communication unit is configured to call a service center of a healthcare provider. This way, the error and/or technical malfunction of the front-end communication unit can be efficiently remedied by remote technical assistance.

According to a further aspect of the present invention, the front-end communication unit is configured to automatically transmit a session ID to a service center of a healthcare provider and to initiate a voice call to a field representative upon completion of a predetermined workflow of the front-end communication unit.

According to a further aspect of the present invention, the front-end communication unit is configured to transmit the data read out from the implantable medical device to a back-end communication unit of the implant communication system. A technician or field representative will then be notified via push message (e.g., SMS, email, etc.) that new data is available. As a result, the technician or field representative logs or dials into the system, looks through the data and then calls the clinic or the physician at the phone number on file (or known to the technician or field representative).

The last two aspects or embodiments considerably simplify and speed up an existing workflow for contacting the field representative for obtaining an analysis of the data transmitted by the front-end communication unit to the back-end communication unit.

The herein described features of the front-end communication unit for an implant communication system are also disclosed for the computer implemented method for operating a front-end communication unit for an implant communication system and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a diagram of an implant communication system according to a preferred embodiment of the present invention; and

FIG. 2 shows a flowchart of a computer implemented method for operating a front-end communication unit for an implant communication system according to the preferred embodiment of the present invention.

### DETAILED DESCRIPTION

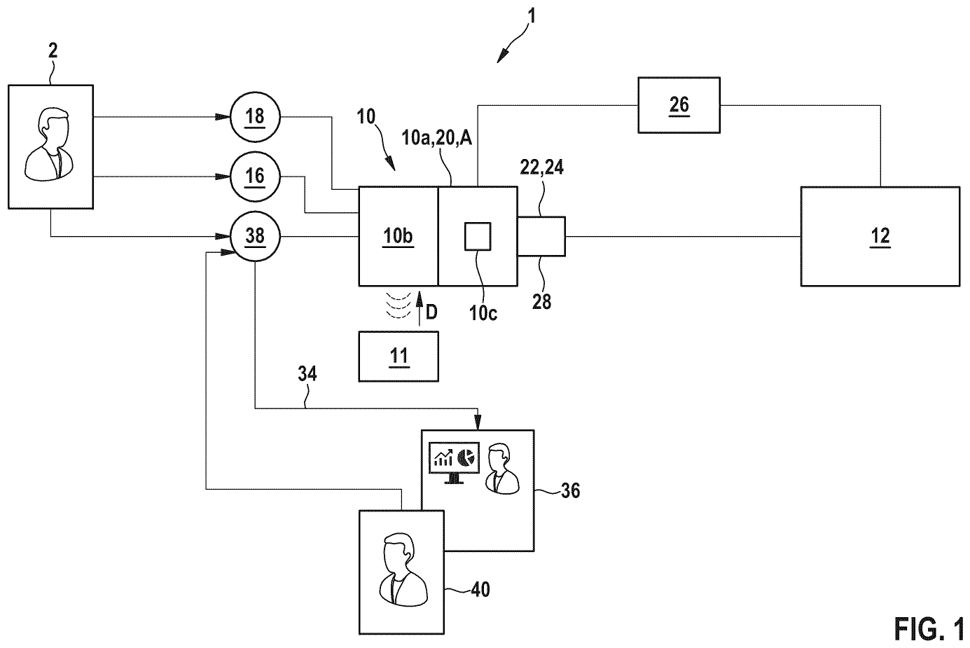

The implant communication system 1 of FIG. 1 comprises a communication interface 10a configured to read out data D from an implantable medical device 11, and a control unit 10b communicatively connected to the communication interface 10a, the control unit 10b being configured to transfer the data D read out by the communication interface 10a to a back-end communication unit 12. Furthermore, the front-end communication unit 10 is configured to receive audio input 16 from a user 2 and provide audio output 18 to the user 2.

Moreover, the front-end communication unit 10 comprises an audio processing unit 20 configured to process the audio input 16 by means of a voice recognition algorithm A, wherein the audio input 16 is configured to process user commands and/or user questions directed to the front-end communication unit 10.

In addition, the audio processing unit 20 is configured, whenever a user commands and/or user questions directed to the front-end communication unit 10 is recognized by the voice recognition algorithm A, to perform a predefined function associated with the user command and/or user question.

The audio output 18 comprises audible status information of the front-end communication unit and/or the implantable medical device 11 and wherein the audio output 18 further comprises audible instructions directed to the user 2 of the front-end communication unit.

The communication interface 10a comprises a mobile interface 22, in particular a LTE and/or 5G-interface, configured to receive incoming voice calls 38 and to initiate outgoing voice calls 38. Furthermore, the communication interface 10a comprises a Bluetooth-interface 24 configured to connect to the implantable medical device 11 and/or to connect to a mobile communication device 26, in particular a smartphone. In addition, the communication interface 10a comprises a WiFi-interface 28 configured to transmit the data D read out from the implantable medical device 11 to a back-end communication unit 12 of the implant communication system 1.

The front-end communication unit 10 is configured, upon a change of information displayed on a screen 10c of the front-end communication unit 10, to generate an audio output 18 corresponding to the information displayed on the screen 10c of the front-end communication unit 10.

The front-end communication unit 10 is configured, upon a status change of the front-end communication unit 10, to generate an audio output 18 corresponding to the status change of the front-end communication unit 10.

The front-end communication unit 10 is configured, when a user interaction with the front-end communication unit 10 is required, to generate an audio output 18 corresponding to the required user interaction with the front-end communication unit 10. The front-end communication unit 10 is further configured to receive and process audio input 16 whenever there is no audio output 18.

In case of an operating error and/or technical malfunction of the front-end communication unit 10, the front-end communication unit is configured to call a service center 36 of a healthcare provider.

Moreover, the front-end communication unit is configured to automatically transmit a session ID 34 to a service center 36 of a healthcare provider and to initiate a voice call 38 to a field representative 40 upon completion of a predetermined workflow of the front-end communication unit.

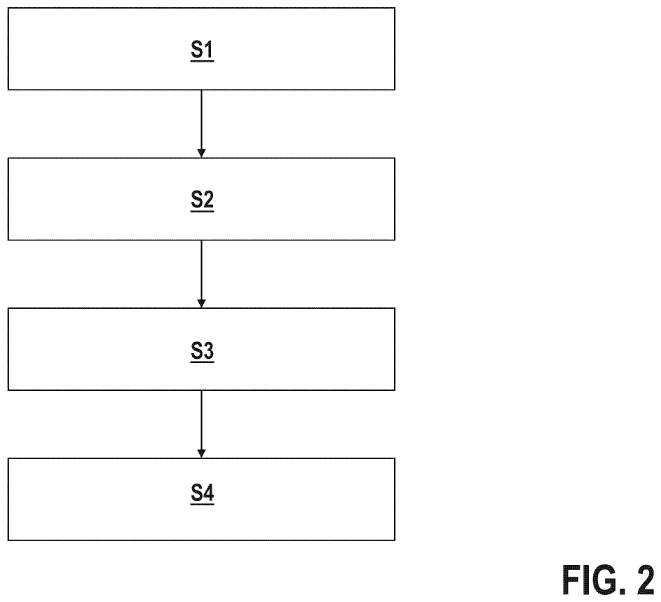

FIG. 2 shows a flowchart of a computer implemented method for operating a front-end communication unit for an implant communication system according to the preferred embodiment of the present invention.

The method comprises reading out S1 data D from an implantable medical device 11 by means of a communication interface 10a and providing S2 a control unit 10b communicatively connected to the communication interface 10a.

Furthermore, the method comprises transferring S3 the data D read out by the communication interface 10a to a back-end communication unit 12 by means of the control unit 10b and receiving S4 audio input 16 and providing audio output 18 by means of the front-end communication unit 10.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
2 user
10 front-end communication unit
10a communication interface
10b control unit
10c screen
11 implantable medical device
12 back-end communication unit
16 audio input
18 audio output
20 audio processing unit
22 mobile interface
24 Bluetooth-interface
26 mobile communication device
28 WiFi-interface
34 session ID
36 service center
38 voice call
40 field representative
A voice recognition algorithm
D Data
S1-S4 method steps

1. Front-end communication unit for an implant communication system comprising: a communication interface configured to read out data from an implantable medical device, and a control unit communicatively connected to the communication interface, the control unit being configured to transfer the data read out by the communication interface to a back-end communication unit, wherein the front-end communication unit is configured to receive audio input and provide audio output, and wherein the communication interface comprises an LTE and/or 5G-interface, configured to receive incoming voice calls and to initiate outgoing voice calls.

2. Front-end communication unit of claim 1, wherein the front-end communication unit comprises an audio processing unit configured to process the audio input by means of a voice recognition algorithm, wherein the audio input is configured to process user commands and/or user questions directed to the front-end communication unit.

3. Front-end communication unit of claim 2, wherein the audio processing unit is configured, whenever a user command and/or user question directed to the front-end communication unit is recognized by the voice recognition algorithm, to perform a predefined function associated with the user command and/or user question.

4. Front-end communication unit of claim 1, wherein the audio output comprises audible status information of the front-end communication unit and/or the implantable medical device, and wherein the audio output further comprises audible instructions directed to a user of the front-end communication unit.

5. Front-end communication unit of claim 1, wherein the communication interface further comprises a mobile interface different from the LTE and/or 5G-interface, configured to receive incoming voice calls and to initiate outgoing voice calls.

6. Front-end communication unit of claim 1, wherein the communication interface comprises a Bluetooth-interface configured to connect to the implantable medical device and/or to connect to a mobile communication device, in particular a smartphone.

7. Front-end communication unit of claim 1, wherein the communication interface comprises a WiFi-interface configured to transmit the data read out from the implantable medical device to a back-end communication unit of the implant communication system.

8. Front-end communication unit of claim 1, wherein the front-end communication unit is configured, upon a change of information displayed on a screen of the front-end communication unit, to generate an audio output corresponding to the information displayed on a screen of the front-end communication unit.

9. Front-end communication unit of claim 1, wherein the front-end communication unit is configured, upon a status change of the front-end communication unit, to generate an audio output corresponding to the status change of the front-end communication unit.

10. Front-end communication unit of claim 1, wherein the front-end communication unit is configured, when a user interaction with the front-end communication unit is required, to generate an audio output corresponding to the required user interaction with the front-end communication unit.

11. Front-end communication unit of any claim 1, wherein the front-end communication unit is configured to receive and process audio input whenever there is no audio output.

12. Front-end communication unit of claim 1, wherein in case of an operating error and/or technical malfunction of the front-end communication unit, the front-end communication unit is configured to call a service center of a healthcare provider.

13. Front-end communication unit of claim 1, wherein the front-end communication unit is configured to automatically transmit a session ID to a service center of a healthcare provider and to initiate a voice call to a field representative upon completion of a predetermined workflow of the front-end communication unit.

14. Computer implemented method for operating a front-end communication unit for an implant communication system comprising the steps of:
reading out data from an implantable medical device by means of a communication interface;
providing a control unit communicatively connected to the communication interface;
transferring the data read out by the communication interface to a back-end communication unit by means of the control unit; and
receiving audio input and providing audio output by means of the front-end communication unit.

15. Computer program with program code to perform the method of claim 14 when the computer program is executed on a computer.
